(19) United States
(12) Patent Application Publication
(10) Pub. No.: US 2016/0199209 A1
(43) Pub. Date: Jul. 14, 2016

(54) LUMBAR SUPPORT STRUCTURE

(71) Applicant: FENG CHING TU, NEW TAIPEI CITY (TW)

(72) Inventor: FENG CHING TU, NEW TAIPEI CITY (TW)

(21) Appl. No.: 14/596,846

(22) Filed: Jan. 14, 2015

(51) Int. Cl. A61F 5/02

(52) U.S. Cl. CPC A61F 5/028

(57) ABSTRACT

A lumbar support structure comprises: a lumbar pad; two circling knee devices, disposed at two sides of the lumbar pad, each possessing a first strip member and a second strip member; two knee pads, movably female-connected with the first strip members, each possessing a penetrating portion with a through channel, the first strip member and the knee pad fabricating a knee-binding structure; two adjustment buckles, each disposed between the first strip member and the second strip member, a front end of the adjustment buckle being fastened with the first strip member, the second strip member penetrating through the adjustment buckle and elongating toward the lumbar pad in order to form an embracing portion, wherein the embracing portion elongates toward the lumbar pad in order to form a movable adjustment band.

### BACKGROUND OF THE INVENTION

1. Field of the Invention

The present invention relates to an ergonomic lumbar support structure that is convenient to operate and can be precisely adjusted.

2. Description of the Prior Art

Modern medical treatment for pelvic misalignment often adopts a lumbar support structure for correction and treatment for daily life. Due to nervous myofascial causing physical discomfort, patients are often inconvenienced by such devices. Besides, because of learning and working, People sitting for several hours each day at work or study may find themselves suffering from backache, back pain, aches, and other discomfort. Such diseases should be carefully treated; otherwise the pelvis, spine, and other parts may be hurt in the long term. As such, proper medical equipment plays an important role in helping people solve these problems. Typical lumbar supporters as shown in FIG. 1 and FIG. 2 have the following components: a lumbar pad 90, with two strips—the longer 91 and the shorter 92, the first strip member 91 penetrates through knee pad 93 to form a knee-binding structure, a front end of one of the first strip members 91 around the knee pad 93 is linked with male buckle 95, the other first strip 91 is linked with female buckle 94, so that the two buckles 94 and 95 are connected with each other to position the lumbar pad 90 around the waist of the patient. In addition, after looping around knee pad 93, strip 91 connects with adjustment buckle 96, wherein the rear of adjustment buckle 96 is firmly connected with the second shorter strip 92. Adjustment buckle 96 has a rib 961, so that the first strip 91 embraces rib 961, elongating to form an adjustment band 911. The lumbar supporter is applied while the patient is seated. To apply the lumbar supporter, first position lumbar pad 90 around the waist. Knee pad 93 is placed on the knee of the patient in order to let the knee-binding structure cover the knee. Finally, to bind the knee tightly, adjust the adjustment band 911 of the first strip 91. Ensure that lumbar pad 90 is tightly attached to the waist, to support the pelvis and lumbar well.

Although the aforementioned lumbar supporter is able to support the pelvis and lumbar, there are several disadvantages. For example, tightness is adjusted by pulling the adjustment band 911 forward, opposite the patient's body, making it difficult to use; further, the pulling direction makes precise control difficult, especially for suitable tightness. In addition, excessive pulling force is applied, in order to re-adjust adjustment band 911, it is necessary to release the first strip 91 embracing rib 961 of adjustment buckle 96, a complicated and inconvenient procedure. Hence, designing a new lumbar support structure that solves the above disadvantages is an important issue.

The inventor of the present invention has studied the flaws of lumbar support structures, and intends to improve the structure for convenience, ergonomics, and ease of use in order to improve the field, helping society.

### SUMMARY OF THE INVENTION

The main objective of this invention is to provide an ergonomic, easily operated lumbar support structure that can be precisely adjusted by redesigning two knee-binding structures and two adjustment bands.

To achieve the above objectives, the lumbar support structure is comprised of: a lumbar pad; two circling knee devices , disposed at the two sides of the lumbar pad, with each of the two circling knee devices having a first strip member and a second strip member, wherein the two pairs of the first strip members and the second strip members are fastened at the lumber pad and elongate from the two sides of the lumbar pad; two knee pads, adjustable female-connected with the first strip members, each of the two knee pads penetrating through the channel, and each of the two first strip members and each of the two knee pads fabricating a knee-binding structure; and two adjustment buckles, each disposed between the first strip member and the second strip member, a front end of each of the adjustment buckles fastened with the first strip member, the second strip member penetrating through the adjustment buckle and elongating toward the lumbar pad in order to form an embracing portion, wherein the embracing portion of the second strip member continuously elongates toward the lumbar pad in order to form a movable adjustment band for adjusting the knee-binding structure.

Accordingly, the first strip member is longer than the second strip member.

Accordingly, the strip members are firmly connected with the sides of their lumbar pads via sewing.

Accordingly, the knee pad (30) has a penetrating portion (31) with a through channel.

Accordingly, two sides of the penetrating portion are attached to the knee pad.

Accordingly, the adjustment buckle has at least one rib for the second strip member to penetrate through.

Accordingly, a top edge, adjacent to the movable adjustment band of the adjustment buckle is an elastic clamp.

Accordingly, the lumbar support structure has two further positioning buckles; each of the two positioning buckles has a male and female positioning buckle that is disposed at the two first strip members respectively.

Accordingly, the knee pad is elongated toward the lumbar pad to form a top knee pad.

### BRIEF DESCRIPTION OF THE DRAWINGS

The objects, spirits, and advantages of the preferred embodiments of the present invention will be readily understood by the accompanying drawings and detailed descriptions, wherein:

FIG. 1 illustrates a schematic structural view of a prior lumbar supporter;

FIG. 2 illustrates a schematic operational view of the prior lumbar supporter;

FIG. 3 illustrates a schematic structural view of a first preferred embodiment of the present invention;

FIG. 3a illustrates a partial enlarged view of the first preferred embodiment of the present invention;

FIG. 3b illustrates another partial enlarged view of the first preferred embodiment of the present invention;

FIG. 4 illustrates a schematic operational view of the first preferred embodiment of the present invention;

FIG. 5 illustrates a schematic structural view of a second preferred embodiment of the present invention; and

FIG. 6 illustrates a schematic operational view of the second preferred embodiment of the present invention.

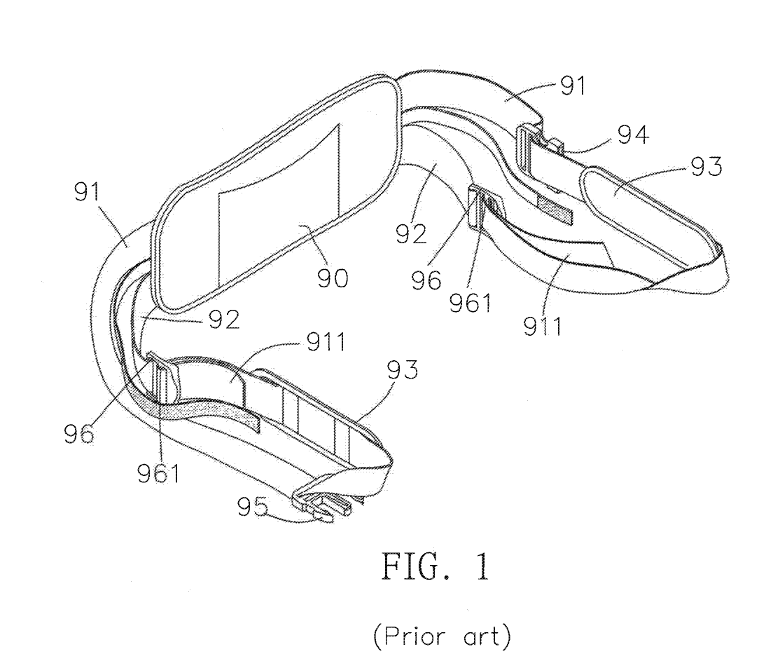

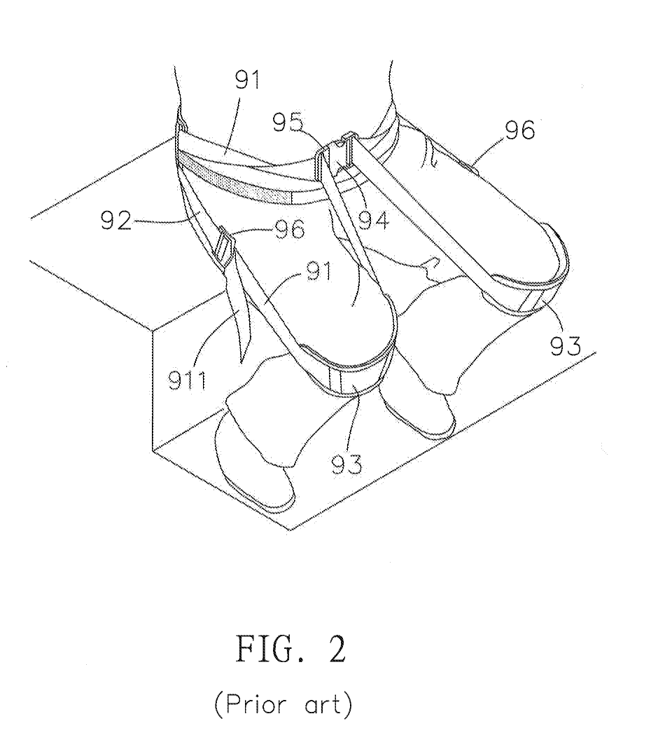

### DETAILED DESCRIPTION OF THE INVENTION

Following preferred embodiments and figures will be described in detail so as to achieve aforementioned goals.

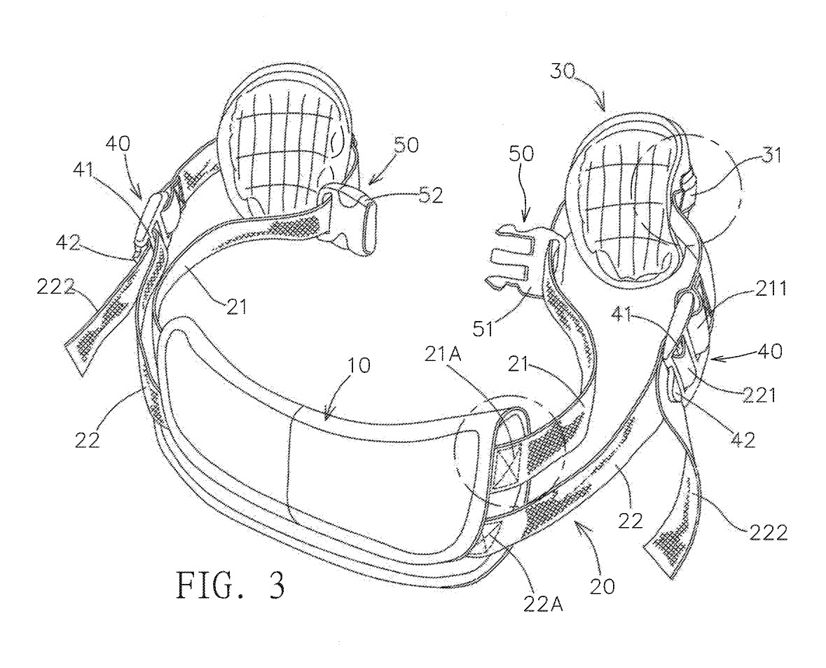

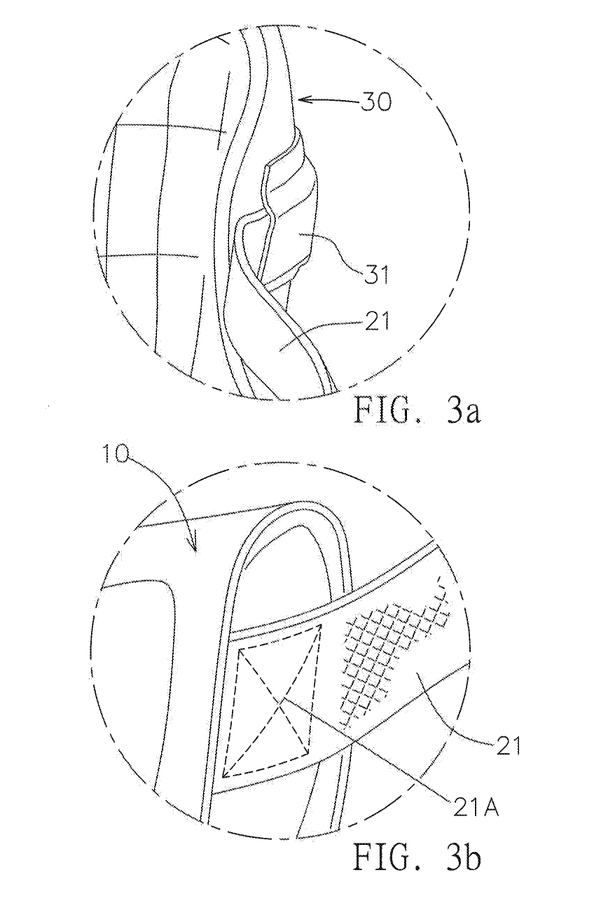

Please refer to FIG. 3, FIG. 3a and FIG. 3b, which illustrate the first preferred embodiment of the current invention's lumbar support structure. The lumbar support structure has a lumbar 10, two circling knee devices 20, two knee pads 30, and two adjustment buckles 40. Lumbar 10 is a rectangular member with internally soft material (such as sponge), and the lateral surface of the lumbar 10 is made of cloth material with permeability for comfort. The two circling knee devices 20 are disposed at two sides of lumbar pad 10; each of the two circling knee devices 20 has a longer first strip member 21 and a shorter second strip member 22, wherein the two pairs of the first strip members 21 and a second strip members 22 are fastened at the lumber pad 10 and elongate from the two sides of the lumbar pad 10. As shown in the figure, the first strip member 21 is firmly connected with the side of the lumbar pad 10 via a sewing portion 21A, and the second strip member 22 is firmly connected with the side of the lumbar pad 10 via a sewing portion 22A. Normally, the first strip member 21 is above the second strip member 22, but not limited thereto. The two knee pads 30 are adjustable female-connected with the first strip members 21, and each of the two knee pads 30 has a penetrating portion 31 with a through channel, wherein two sides of the penetrating portion 31 are attached to knee pad 30. The first strip member 21 penetrates through the penetrating portion 31 so as to let the knee pad 30 female-connect with the first strip member 21. The purpose of knee pad 30 is to protect the patient's knee, and cooperates with the first strip member 21 to fabricate a knee-binding structure (not numbered).

Each of the two adjustment buckles 40 is between the first strip member 21 and the second strip member 22 in order to adjust the knee-binding structure, wherein the adjustment buckle 40 is shaped as a rib, but not limited thereto. The front end of each of the adjustment buckles 40 is fastened with the longer first strip member 21, that is, the first strip member 21 penetrates through the knee pad 30, and then a fastening end 211 of the first strip member 21 is firmly connected with the adjustment buckle 40, wherein the adjustment buckle 40 has at least one rib 41 for the second strip member 22 penetrating through and elongating toward the lumbar pad 10 in order to form an embracing portion 221. The embracing portion 221 of the second strip member 22 continuously elongates toward the lumbar pad (10) in order to form a movable adjustment band 222; further, a top edge, adjacent to the movable adjustment band 222, of the adjustment buckle 40 is a elastic clamp 42 that is capable of suitably holding the adjustment band 222.

In addition, the lumbar support structure further has two positioning buckles 50, each of the two positioning buckles 50 has a male positioning buckle 51 and a female positioning buckle 52 that are disposed at the two first strip members 21 respectively. The male positioning buckle 51 or the female positioning buckle 52 is between the lumbar pad 10 and the knee pad 30. The male positioning buckle 51 and the female positioning buckle 52 are fastened with each other so as to let the first strip member 21 be positioned on the lumber of a patient.

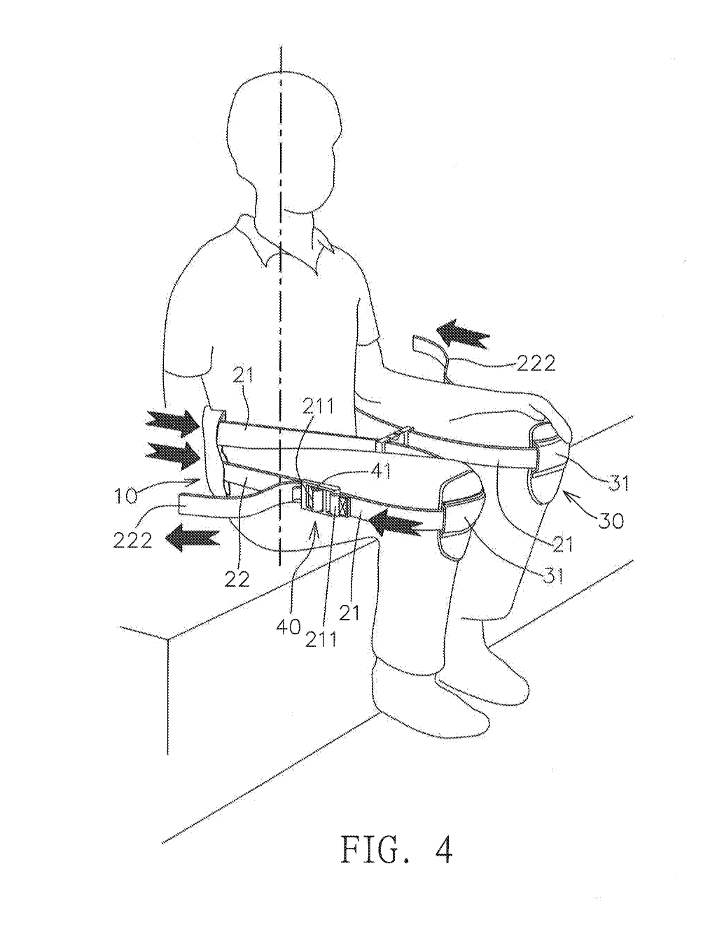

Please refer to FIG. 4, which illustrates the present invention's lumbar support structure in an operational state. As shown in the figure, lumbar pad 10 is firstly positioned on the lumbar, the first strip member 21 and the second strip member 22 are fastened with each other via the adjustment buckle 40, the knee-binding structure (the first strip member 21 plus knee pad 30) is embraced or female-connected with the knee of the patient, and the knee is continuously covered by knee pad 30. After pulling adjustment band 222 of the second strip member 22 backward, adjustment band 222 also pulls the first strip member through the adjustment buckle 40 in order to let the knee-binding structure tie the knee and lumbar pad 10 tightly to the lumbar as well. As seen, the pelvis and lumbar are supported simultaneously.

The present invention adopts the shorter second strip member 22 to movably connect with adjustment buckle 40 in order to pull adjustment band 222 backwardly. This is to facilitate for precise operations in order to provide the best support to the lumbar of the patient.

While in operation, the two legs of the patient are able to move around, so as to relieve, release the spine and pelvic, muscular fascia; in addition, the two knees move back and forth respectively. When one side of the knee is pushed forward, the hip on the same side will also be moved; when the knee on the other side is pushed backward, the surrounding fascia of the ipsilateral hip is squeezed as well. Therefore, to repeat such actions may relax the fascia surrounding the hip and the intestinal lace of the lower abdomen. This will help alleviate and relieve chronic backache, back pain, sore logs, or pelvic misalignment causing taut muscular fascia. Further, when sitting in stationary or general condition, poor posture can be adjusted to prevent backache and back pain. If the present invention is applied in meditation, a triangle-force among two knees and pelvic may be balanced, and meditation can be more relaxed and longer lasting.

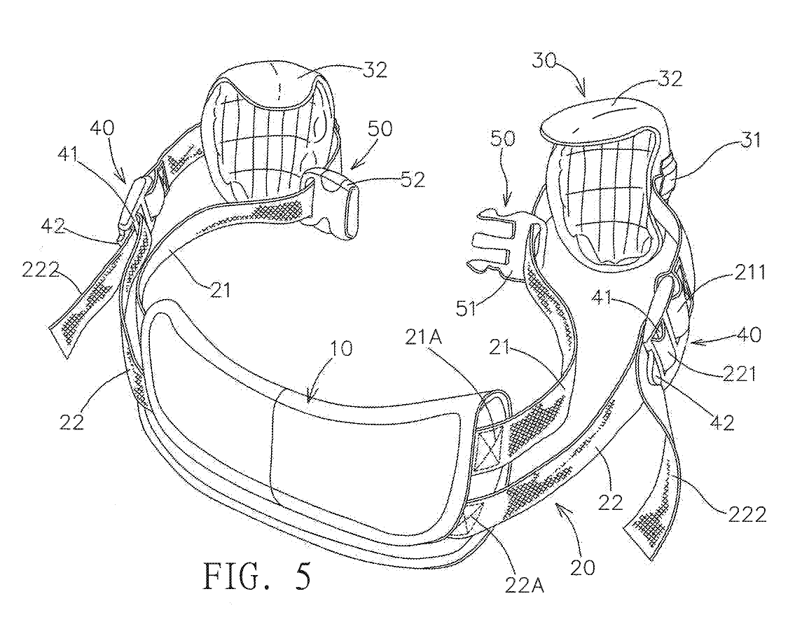

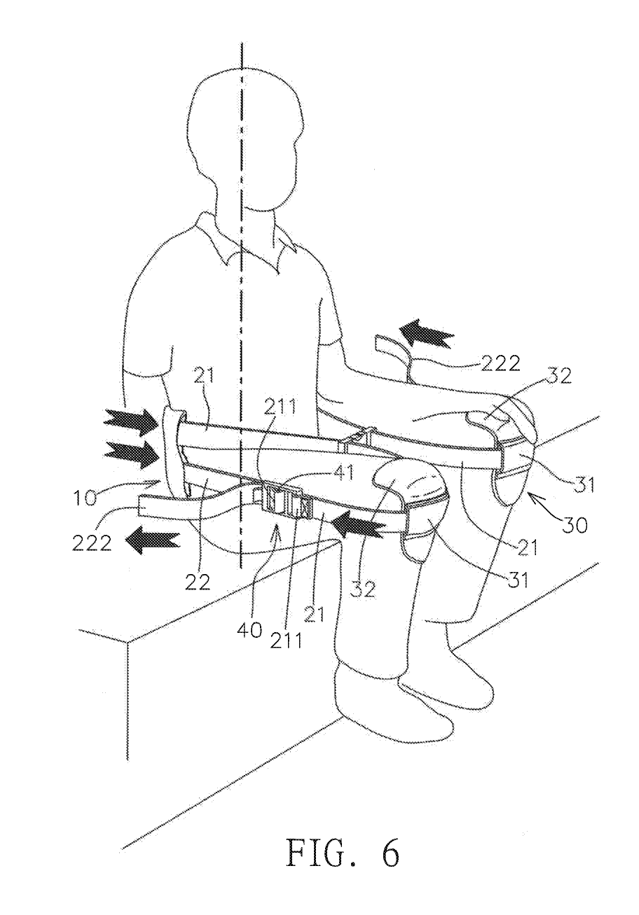

Referring to FIG. 5 and FIG. 6, which illustrate a second preferred embodiment of the lumbar support structure of the present invention. The second preferred embodiment is a variant based on the first preferred embodiment, and the differences between the first preferred embodiment and the second preferred embodiment are listed below: the knee pad 30 further has a top knee pad 32, wherein the top knee pad 32 is formed by elongating knee pad 30 toward the patient. As can be seen, such arrangement is protective to the knees. For a long-term patient or a patient with poor knees, the two hands of the patient resting upon the two top knee pads 32 while in operation will help keep the knees warm.

As described above, the structure adopts the knee-binding structure and the adjustment band to facilitate precise ergonomic operations in order to improve convenience and speed.

Although the invention has been described and illustrated with reference to particular embodiments, the principles involved are suitable for use in numerous other embodiments that will be apparent to persons skilled in the field. This invention is, therefore, to be limited only as indicated by the scope of the appended claims.

What is claimed is:

1. A lumbar support structure, comprising:
a lumbar pad (10);
two circling knee devices (20), disposed at two sides of the lumbar pad (10), each of the two circling knee devices (20) having a first strip member (21) and a second strip member (22), wherein the two pairs of the first strip members (21) and a second strip members (22) are fastened at the lumber pad (10) and elongate from the two sides of the lumbar pad (10);
two knee pads (30), movably female-connected with the first strip members (21), each of the two knee pads (30) having a penetrating portion (31) with a through channel, each of the two first strip members (21) and each of the two knee pads (30) fabricating a knee-binding structure; and
two adjustment buckles (40), each of the two adjustment buckles (40) being disposed between the first strip member (21) and the second strip member (22), a front end of each of the adjustment buckles (40) being fastened with the first strip member (21), the second strip member (22) penetrating through the adjustment buckle (40) and elongating toward the lumbar pad (10) in order to form an embracing portion (221), wherein the embracing portion (221) of the second strip member (22) continuously elongates toward the lumbar pad (10) in order to form a movable adjustment band (222) for adjusting the knee-binding structure.

2. The lumbar support structure according to claim 1, wherein the first strip member (21) is longer than the second strip member (22).

3. The lumbar support structure according to claim 2, wherein the first strip member (21) is firmly connected with the side of the lumbar pad (10) via a sewing portion (21A), and the second strip member (22) is firmly connected with the side of the lumbar pad (10) via a sewing portion (22A).

4. The lumbar support structure according to claim 1, wherein two sides of the penetrating portion (31) are attached to the knee pad (30).

5. The lumbar support structure according to claim 1, wherein the adjustment buckle (40) has at least one rib (41) for the second strip member (22) to penetrate through.

6. The lumbar support structure according to claim 1, wherein a top edge, adjacent to the movable adjustment band (222), of the adjustment buckle (40) is a elastic clamp (42).

7. The lumbar support structure according to claim 1 further having two positioning buckles (50), each of the two positioning buckles (50) has a male positioning buckle (51) and a female positioning buckle (52) that are disposed at the two first strip members (21) respectively.

8. The lumbar support structure according to claim 1, wherein the knee pad (30) is elongated toward the lumbar pad (10) to form a top knee pad (32).
